(19) United States
(12) Patent Application Publication
(10) Pub. No.: US 2009/0156889 A1
(43) Pub. Date: Jun. 18, 2009

(54) METHOD OF INDUCING SLEEP

(76) Inventor: Maheswar R. Mikkilineni, Washington, DC (US)

Correspondence Address: STEPHEN CHRISTOPHER SWIFT;SWIFT LAW OFFICE, SUITE 600, 1800 DIAGONAL ROAD, ALEXANDRIA, VA, 22314-2840, US

(21) Appl. No.: 12/259,285

(22) Filed: Oct. 27, 2008

Related U.S. Application Data
(63) Continuation-in-part of application No. 11/999,349, filed on Dec. 5, 2007, now abandoned.
Application No. 11/999,349 is a continuation-in-part of application No. 11/449,519, filed on Jun. 8, 2006, now abandoned.

(51) Int. Cl. A61M 21/02

(52) U.S. Cl. 600/27

(57) ABSTRACT

Method to create bliss in mind and induce sleep, using devices that are worn on the nose and around the diaphragm. The steps included in the method to induce sleep are: 1. Calming the mind of intrusive thoughts, or decreasing excited-signals in the brain, to prepare for a sleep cycle by concentrating upon one principal thought, on the device or breath. 2. Splitting the brain to activate the master-clock in the brain, and induce a sleep cycle, by concentrating upon two or more principal thoughts, on the devices or breath and movement. The steps included to practice-learn the method are:
1. Identify reason for intrusive thoughts and select appropriate principal thoughts, if device did not yield a quick result.
2. Practice-learn to calm the mind using one principal thought.
3. Practice-learn to split the brain and fall-asleep using two or more principal thoughts at the same time.

### CROSS REFERENCE TO RELATED APPLICATION

This application is a Continuation-In-Part of Regular Utility patent application Ser. No. 11/999,349, filed Dec. 5, 2007, which was a Continuation-In-Part of Regular Utility patent application Ser. No. 11/449,519, filed Jun. 8, 2006, now abandoned, and which are incorporated herein by reference.

### BACKGROUND OF THE INVENTION

1. Field of the Invention

The present invention relates to methods for inducing sleep, without the use of drugs.

2. Description of the Prior Art

The brain is the control center of the body. It controls thoughts-signals, senses, memory and function of cells-organs. Survival depends on a properly functioning brain to regulate the functions of the organs, so that the heart pumps blood, the lungs breath oxygen-air, and the digestive system assimilates water-food.

The brain requires a sleep-cycle, deep sleep (DS) and rapid eye movement (REM) sleep, of about eight hours in a 24-hour day to function properly. A recent study (see REF.-2) at Harvard Medical School and University of California concludes—
“ . . . a lack of sleep causes the brain's emotional centers to dramatically overreact . . . (with) psychiatric disorders . . . (and) fractures the brain mechanisms that regulate key aspects of mental health . . . and, sleep appears to restore emotional brain's circuits . . . ”

Thus, sleep-problems play a key-role in a large number of brain disorders. For example, strokes and asthma attacks tend to occur more frequently due to changes in hormones, heart rate, breathing rate, and other changes associated with sleep. Neurons that control sleep interact closely with the immune system—sleep may help body conserve energy that the immune system needs to mount an attack. Sleep problems occur in almost all people with mental disorders, including those with depression and schizophrenia. A person with depression is often awake in the night and unable to get back to sleep. Extreme sleep deprivation can lead to a seemingly psychotic state of paranoia and hallucinations in an otherwise healthy person, and disrupted sleep can trigger episodes of mania-agitation and hyperactivity, to a person with manic depression.

The National Sleep Foundation's (NSF) sleep in America poll found that 74% of American adults experience sleep problems a few nights a week or more, 39% get less than seven hours of sleep each weeknight, and more than one in three (37%) are so sleepy during the day that it interferes with daily activities. An article ‘Counting sheep no aid to insomnia’ (see REF.-5) published in January 2002, on the findings of Oxford study on sleep, reported that—

“1 in 10 suffer from chronic insomnia and it is estimated that sleeplessness costs the US economy $35 billions a year . . . ”

The sleep problems arise from changes in the brain regions and neurons that control sleep, or from the drugs used to control other disorders. Once a sleep problem develops, it can add to a person's impairment and cause confusion, frustration, or depression. Sleep-problems are common in many other disorders as well, including Alzheimer's disease, stroke, brain injury and cancer. In the December 2007 issue of The Lancet Oncology, the International Agency for Research on Cancer, the cancer arm of the World Health Organization, published the results of its analysis of an expert panel, and reports—

“Scientists suspect that shift work (night-work) is dangerous because it disrupts the circadian rhythm, the body's biological clock. The hormone melatonin, which can suppress tumor development, is normally produced at night. Light shuts down melatonin production, so people working in artificial light at night may have lower melatonin levels, which scientists think can raise their chances of developing cancer. Sleep deprivation may also be a factor. People who work at night are not usually able to completely reverse their day and night cycles. Not getting enough sleep makes your immune system vulnerable to attack, and less able to fight off potentially cancerous cells. Certain processes like cell division and DNA repair happen at regular times. But if the body needs to do something at an unusual time—like produce insulin in the middle of the night to help digest food—that can set off a chain reaction of biological mistakes. The problem is re-setting your body's clock. Anyone whose light and dark schedule was frequently disrupted—including frequent long-haul travelers or insomniacs—could face the same increased cancer risks. The balance between light and dark is very important for your body, and make sure to sleep in a darkened room. Just get a dark night's sleep.”

Circadian rhythms are regular changes in mental and physical characteristics that occur in the course of a day (circadian is Latin for “around a day”). Most circadian rhythms are controlled by the body's biological “master-clock” in the brain. The pattern of waking during the day when it is light and sleeping at night when it is dark is a natural part of life's circadian rhythm. Only recently have scientists begun to understand sleep alternating and how it is related to daylight and darkness. A key factor that regulates human sleep is the exposure to light or to darkness. Exposed light reaches photoreceptors in the retina of eye, and creates signals to stimulate a nerve pathway which travel along the optic nerve from retina to an area in the brain called ‘thalamus-hypothalamus’. There, a special center called the supra-chiasmatic nucleus (SCN) initiates signals to other parts of the brain, including sending excited-signals into the cortex which keeps a person wide awake. This SCN is called the “master-clock”, and it is actually a pinhead-sized brain structure that contains about 20,000 neurons.

The SCN works to set a regulated pattern of sleep-cycle. Once exposed to the first light each day, the clock begins performing functions like raising body-temperature, rate of heart-beat and breathing, and releases stimulating hormones like cortisol. The SCN delays the release of other hormones like melatonin (dopamine), which are associated with sleep onset, until many hours later when darkness returns. The signals from SCN travel to several brain regions, including the pineal gland, which responds to light-induced signals by switching off production of the hormone melatonin. The body's level of melatonin normally increases after darkness falls, making a person feel drowsy. The SCN governs functions that are synchronized with the sleep-wake cycle, including body temperature, hormone secretion, urine production, and changes in blood pressure, heart rate and breathing etc. Because sunlight or other bright lights can reset the SCN, the biological cycles normally follow the 24-hour cycle of the sun. Disruptions in circadian rhythms increase the risk of heart problems, digestive disturbances, and emotional and mental problems, all of which may be related to sleep-problem.

Melatonin is a natural hormone made by the human body's pineal gland, which is a pea-sized gland located in the brain close to ‘thalamus’. During the day the pineal gland is inactive. When the sun goes down and darkness occurs, the pineal is supposed to be turned on by an active SCN, to begin producing melatonin and release into the blood.

When the SCN works as it should, it results in inviting sleep to the brain. Melatonin levels in the blood stay elevated for about twelve hours, all through the night, before the light of a new day when it falls back to low daytime levels. Besides adjusting the timing of the body's clock, bright light has another effect—it directly inhibits the release of melatonin.

Currently, there are several prescription drugs available to aid sleep. They can shorten the time it takes to fall asleep and reduce awakenings, which adds to total time spent asleep. Possible side effects include feeling tired or drowsy the next day, memory loss, headache and problems with performance. Prescription sleeping pills can cause strange and potentially dangerous side effects. Those side effects can include dangerous allergic reactions and bizarre behaviors such as sleep eating, walking and driving, in which a person will drive a car while not fully awake and has no memory of doing so.

At this stage, science is trying to find a way to implant a new ‘light switch’ in the brain (see REF.-4) for treating mental-disorder(s), and/or to control sleep-wake state of the brain (see REF.-3). Science is unable to locate and ‘repair’ the ‘light switch’ which exists in the brain-stem and is unable to function. The method or process in the instant invention is able to ‘repair’ the ‘light switch’ and make it function without an implant or use of drug.

Published articles relevant to the invention are cited, and copies of the references cited are attached:
REF.-1: Concept of the dominant hemisphere, p. 719, Medical Physiology, Guyton & Hall, 11^{th} edition.
REF.-2: Sleepless Nights make Grumpier Brains, October 2007.
REF.-3: Sleep-Wake controls identified, July 2007.
REF.-4: Beam of light-flips a switch in Brain, August 2007.
REF.-5: Counting sheep no aid to Insomnia, Oxford study.
REF.-6: Insomnia Tip #20, Counting Sheep, from http://www.well.com/-mick/insomnia.12.html, printed on Jun. 21, 2008.
REF.-7: Copper Meditation for the 21st Century: Part 1, NEW Worlds Issue: NW034, Dr. John Mumford, 2002, printed Jun. 21, 2008.
REF.-8: Yoga & Insomnia, Llewellyn Journal, by Thomas Polkhome, Aug. 22, 2005, from http://llewelynjournal.com/print.php?id=880.
REF.-9: Script for Guided Imagery, The Wellness Community, by Harold H. Benjamin, Ph.D., ©1994, from http://www.twc-wla.org/images/GuideImg_files/GuideImg.htm, printed Jun. 20, 2008 available December 2004 per wayback machine.
REF.-10: Printout from Internet (unknown URL).
REF.-11: Printout from Internet (unknown URL).
REF.-12: Guyton & Hall, Medical Physiology, 11th Edition, p. 729.
REF.-13: Guyton & Hall, Medical Physiology, 11th Edition, p. 730.

A careful review of present scientific knowledge in the matter (see REFS. 1 to 4) reveals solid proof in support of the instant invention that this method is ‘ahead’ of its time, as seen from the findings of the science summarized below:
That brain has two hemispheres and one is dominant;
That the two hemispheres communicate with each other, one thought at a time;
That any interference from two or more thoughts in the mind at the same time creates havoc with mental-thoughts and motor-responses in the brain, and the two hemispheres stop communicating—a split brain (see REF.-1).
That split brain causes the light switch ‘flip’ in the brain-stem's recticular activating system (see REF.-3) to activate the master-clock and make brain fall-asleep or help cure depression or mental-disorder(s) etc. (see REF.-2).
That the science is struggling to introduce a ‘protein or gene’ into the brain from outside to ‘fix’ depression and/or control sleep-wake state with a ‘flip’ of the light switch;
That the science is unable to locate and ‘repair’ the light switch that exists in the brain, but does not work.
That the science is looking for a solution elsewhere (see REFS. 3 & 4), but not within where the solution lies.
That the instant invention looked for a solution within and found it, before the science can ever find a solution, as safe as the instant process!

The finding from Oxford study, or the article known as CNN.com—‘Counting sheep no aid to Insomnia’ (REF.-5), is not identical to the instant invention, although the ‘scene’ or ‘counting sheep’ may had two or more elements in it; the participants in that study utilized one-thought at a time and did not know how to bring-in two or more thoughts at the same time into the mind. The Oxford study, however, is in agreement with the instant invention on one aspect, the ‘selection of an object, location or action’ to generate positive-signals in the mind. The study found that a thought to ‘imagine a scene’ works, but a thought to ‘count sheep’, the traditional cure of 19^{th} century, “does not work”.

REF.-6, Insomnia Tip #20—Counting Sheep, as in REF.-5, does not discuss how to bring into the mind two or more thoughts at the same time, as in the instant invention.

REF.-7, Copper Meditation for the 21st Century, does not discuss how to bring into the mind two or more thoughts at the same time, as in the instant invention.

REF.-8: Yoga & Insomnia, does not discuss how to bring into the mind two or more thoughts at the same time, as in the instant invention.

REF.-9: Script for Guided Imagery, does not discuss how to bring into the mind two or more thoughts at the same time, as in the instant invention.

REF.-10 is a schematic view of the two hemispheres of the brain.

REF.-11 is a diagrammatic sectional view of right-hemisphere of the brain looking from inside: it identifies the location of sustantio nigra which produces melatonin, and the pathways of melatonin into thalamus where it decreases the signal-activity going into the cortex; and it identifies the location of brain-stem which turns-off the light switch (when split-brain occurs) allowing serotonin to take pathways into thalamus-hypothalamus and cortex to activate the master-clock and begin the sleep cycle.

REF.-12 is a diagrammatic sectional view of left-hemisphere of the brain looking from inside: it identifies the locations for excitatory and inhibitory areas in the brain-stem. The upper brain-stem sends signals into thalamus to get them excited; then, thalamus sends those excited signals into appropriate regions of the brain-cortex. The lower brain-stem cuts off the signals going out of upper brain-stem (when split-brain occurs) and flips off the light switch.

REF.-13 is a diagrammatic view showing the key parts in the brain where melatonin and serotonin are made.

The invention as a whole is to benefit a human being, a user of the process where a series of steps or thoughts are implemented using device(s) for practical application or to transform brain-neurons into a different state to function. The claims are directed to cure a natural-phenomena of ‘intrusive-thoughts’ streaming into a sick-brain, a physical object that gets “repaired and transformed” into a different state in the process. The “process” is patentable under 35 USC § 101, see Parker 437 U.S. 584, Funk Bro. Seed Co. 333 U.S. 127, and Mackay Radio & Tel. Co. 306 U.S. 86, where the court said:

“ . . . when a claim containing a mathematical formula implements or applies that formula in a structure or process which when considered as a whole, is performing a function (transforming or reducing an article to a different state) which the patent laws were designed to protect . . . ”

In the instant invention, the generation of positive-signals from principal thoughts is a mathematical formula, tuning in the new-signals to flip off the light switch in the brain-stem is the application of the formula, and making the brain perform a function upon repair-transformation into a different state is a process as a whole which the patent laws must protect. The claim of beneficial result is assured and repeatable to a person who learned the skill of the art. The “process” involves (1) generating positive-signal data in the mind using principal thoughts, like writing a code-data on a piece of paper using thoughts; (2) inputting that signal data in the mind into the brain-algorithm, like inputting data from a piece of paper into a machine-algorithm in the computer; (3) tuning in the signal data in the brain-algorithm, like processing data in the machine-algorithm or computer-code; and (4) repairing and transforming brain-algorithm or the neurons to function and provide the desired output, a sleep-cycle, like repairing and transforming the machine-algorithm to function and provide a desired output. That's how the ‘process’ in Microsoft's machine-algorithm came into existence to function: Microsoft ‘repaired and transformed’ the ‘old-IBM’ machine-algorithm or process-code to make it work using new-data signals generated by a thought ‘process’, as in the instant invention. When the thought-process in Microsoft is patented, then, the process in the instant invention should be patentable.

The invention is not a game of ‘abstract functions’ or a ‘mental-exercise’ for fun. It is a “process” to cure mental-disorder(s) with no adverse affect. Thought is not an abstract function; it is the key to everything a human being can do. A set of thoughts can generate code-data to be written for use in a computer; a related set of thoughts can input that code-data to be processed in the computer; and, yet another related set of thoughts can make the produced output from that code-data work for benefit. Similarly, a set of thoughts in this instant invention generate data in the mind, input that data into a non-working algorithm in the brain, and keep processing that data until the algorithm accepts it and become functional, and able to produce a desired output: a sleep-cycle. The ‘repair and transformation’ of brain to produce a desired output is not a natural phenomenon, as it can only be achieved using the process described in the instant invention.

An abstract idea or even a series of mental-steps (or thoughts) is patentable under 35 U.S.C. § 101, if it can create a ‘novel and useful structure or process’ for a useful end. See Parker 437 U.S. 584, Funk Bro. Seed Co. 333 U.S. 127, and Mackay Radio & Tel. Co. 306 U.S. 86, where the court said-

“A process is not unpatentable simply because it contains a law of nature or algorithm (as in the instant invention—thoughts in mind or algorithm in brain) . . . if there is to be an invention (even from an abstract idea), it must come from the application of the law to a new and useful end . . . while a scientific truth is not a patentable invention, a novel and useful structure or process created with the aid of knowledge of scientific truth may be . . . (when the process performs a function)”.

Split brain, physical result or output or other confirmation to the user that something has occurred as a result of this method can be verified by a monitor that records the output of signal-activity in the brain when awake with streaming intrusive thoughts and when asleep. The output will confirm the result, and the person will know of the beneficial result upon awakening from a restful sleep. That is true in many other instances, such as the electric-shock, radiation or oxygen therapy, where the person cannot see the input signals or the process of ‘repair and transformation’ of cells or the output of a different physical state. The final result achieved by the method or process in the present invention is useful, tangible and concrete, and the result is repeatable.

U.S. Pat. No. 5,318,503, issued on Jun. 7, 1994, to Robert F. Lord, discloses a method and apparatus for auditory and olfactory relaxation, using a headset that both generates sound and diffuses fragrance. The instant invention is distinguishable, in that it requires no similar apparatus, and it induces sleep rather than merely relaxation.

U.S. Pat. No. 5,599,274, issued on Feb. 4, 1997, to Nusa Widjaja and Robert W. Fish, discloses a trophotropic response system, which uses both light and sound to relax the user. Again, the instant invention is distinguishable, in that it requires no similar apparatus, and it induces sleep rather than merely relaxation.

U.S. Pat. No. 6,641,522, issued on Nov. 4, 2003, to Joseph August, discloses an apparatus for projecting “biophilic” natural landscape scenes, which is not required by the instant invention.

U.S. Pat. No. 6,641,523, issued on Nov. 4, 2003, to Matthew Ashenden, discloses an apparatus for reducing stress that combines music and words, which is not required by the instant invention.

None of the above inventions and patents, taken either singly or in combination, is seen to describe the instant invention as claimed to induce sleep.

### SUMMARY OF THE INVENTION

The present invention has three parts: (1) Prasanti-loka (Sanskrit for “bliss in mind”) or method to induce sleep, (2) teaching the method with devices, and (3) the devices. It is a process to create peace in mind to induce sleep, and a method to fall-asleep by learning to use the process-algorithm in the brain transform brain-neurons into a different-physical state and produce melatonin and serotonin on its own, without the use of drugs. The process with the devices will assist the user practice and learn the skill of the art or the method to achieve useful, concrete and tangible result or benefit. The principal concept of the method can be summarized as: concentrate upon principal thoughts to calm mind, and split brain to turn-off the light switch in the brain-stem and activate master-clock (SCN) to fall-asleep, and teach persons to learn the skill of the art. The first step is to calm the mind of intrusive thoughts to prepare for a sleep cycle by calmly visualizing in the mind one principal thought upon the nose-device or the breath (or any object, location or action etc.) The second step is to split the brain to activate the master clock (SCN) and induce a sleep cycle by calmly-equally visualizing in the mind at the same time two or more principal thoughts upon the nose-device or the breath (inhale-exhale) and upon the diaphragm-device or the movement of diaphragm (or any object, location or action etc.) The invention includes a process to practice and learn the method with devices. There are two devices in this invention—one to use on the nose, and the other to use on the diaphragm, during practicing the process to learn the skill of the art.

The practice to breath with the nose-device and the diaphragm-device makes one breath freely and deeply more oxygen into the lungs, and into the blood-stream to alleviate pain in the toes and elsewhere or hearing-loss, loss of eye-sight, and the problems due to excess gas in the stomach or difficulty in urination etc., caused by peripheral neuropathy.

It is a principal object of the invention to calm the mind and split the brain to induce sleep in a person who otherwise is unable to go to sleep naturally.

It is an object of the invention to alleviate stress, anxiety, anger, mental-distress, depression, mental-disorders or neurodegenerative and neurological disorders, and health problems due to high blood pressure, hypertension, hot flashes, obesity and cancer.

It is an object of the invention to provide a method that any person, irrespective of culture, religion, nationality, sex, wealth or lack of it (poor) can use and derive benefit from it in an inexpensive, dependable and effective way, without drugs.

It is an object of the invention to provide improved elements and arrangements thereof in the devices for the purpose described which is inexpensive, dependable and fully effective in accomplishing the intended purpose.

These and other objects of the present invention will become readily apparent upon further review of the following specification and drawings.

### BRIEF DESCRIPTION OF THE DRAWINGS

FIG. 1 is a flowchart summarizing the steps of the process with devices involved in the instant invention.

FIG. 2 is a front-view of the nose-device when put on the nose.

FIG. 3 is a plan-view of the nose-device before put on the nose.

FIG. 4 is a plan-view of the diaphragm-device before put on the diaphragm.

FIG. 5 is a top-view of the diaphragm-device when put on the diaphragm.

### DETAILED DESCRIPTION OF THE PREFERRED EMBODIMENT

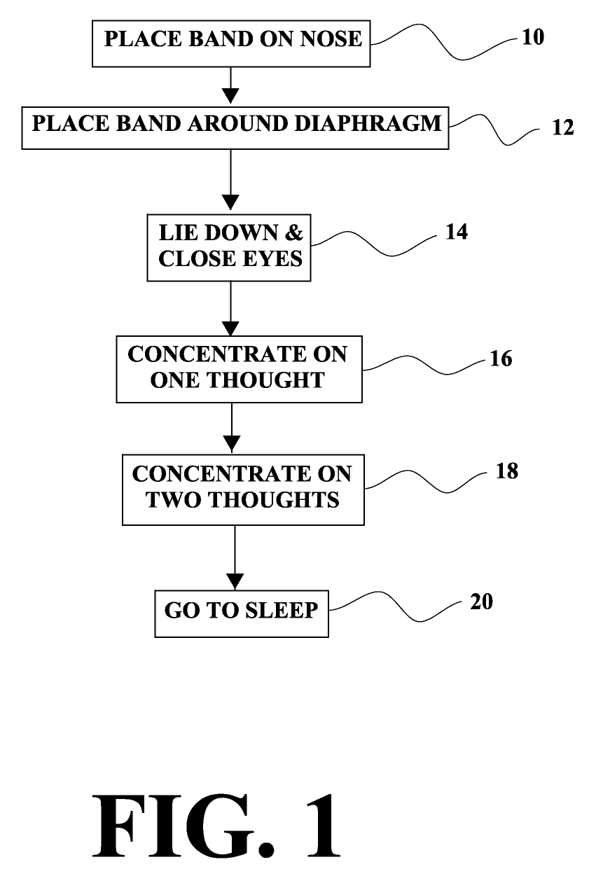

The present invention is a method of inducing sleep, that is named Prasanti-loka (Sanskrit for “bliss in mind”). The steps are summarized in the flowchart of FIG. 1. The user places a nose-band on his (or her) nose 10. The user also places a diaphragm-band on his (or her) diaphragm 12. The user lies down and closes eyes 14. The user first concentrates on one thought 16. The user then concentrates on two or more thoughts 18. As a result, the user then goes to sleep 20.

The process (or art, method) with devices consists of a series of steps or positive thoughts in the mind which generate positive signals, input the signals, and processes the algorithm in the brain to create bliss, and induce a sleep-cycle, deep-sleep (DS) and rapid-eye-movement sleep (REM). The positive signals generated in the mind will tune the brain off of excited negative-signals generated by the natural phenomena of streaming intrusive thoughts into the mind as a result of anxiety, anger, depression or mental-disorder(s). The processing of positive signals in the algorithm ‘repairs and transforms’ the brain into a different-physical state and restores the brain's ability to produce melatonin and serotonin to activate the master clock in the brain for a sleep-cycle as the output, by flipping off the ‘light-switch’ in the brain-stem.

The series of steps in the method tune the brain, first by decreasing the frequency of excited-signals in the brain-cortex, and then cutting off the excited negative-signal activity across the neurons in the recticular activating system of the brain-stem. One initiates tuning of the brain by concentrating the mind upon one principal thought, for example, the ‘device on the nose or inhale-exhale of the breath’, which helps to calm the mind by decreasing the frequency of excited-signals in the brain-cortex. The process generates a set of positive-signals in the mind, inputs that data into the process-algorithm in the brain, tunes the new-signals replacing the old-signals in the brain-cortex, and decreases the frequency of excited-signals in the cortex to cause calm mind and brain produce melatonin, a sleep inviting hormone.

Continue the tuning of the brain by concentrating the mind calmly-equally upon two or more principal thoughts at the same time, for example, the ‘device on the nose or inhale-exhale of the breath and the device on the diaphragm or movement of diaphragm’, which helps to cut off all excited-signals coming into the brain. The process generates two (or more) sets of positive-signals in the mind, inputs that data into the process-algorithm in the brain, tunes the two sets of new-signals to cause an ‘interference created havoc in the brain's motor-responses’ which splits the two hemispheres of the brain to stop them from communicating with each other, and makes the ‘light switch’ in the brain-stem to flip off and cut all excited-signals coming into the brain, which helps the brain produce serotonin to activate the master-clock and put the brain into a sleep-cycle.

The brain that was unable to fall-asleep before begins to function, having been ‘repaired and transformed’ into a different-physical state in the process. The instant invention is ‘ahead’ of its time as the present knowledge of science is looking to implant a new ‘light switch’ in the brain to flip on-off, and treat mental-disorder(s) and/or control the sleep-wake state of the brain. Science is unable to locate and ‘repair’ the ‘light switch’ which exists in the brain-stem. The instant invention is able to ‘repair’ that ‘light switch’ which failed or is not working.

In this method to induce sleep, a person is required to put on the devices, lay in bed (or sofa) and close eyes at the start of step 14 and keep them closed through step 20 to cut off incoming light into the eyes and get dark-night's sleep. The person is required to generate positive-signals in the mind and input that new data into the process-algorithm at step 16 by visualizing one principal thought about any object-thing, location-spot, or action-item that exists within or without, for example the ‘device on the nose or inhale-exhale of the breath’. The person is required to generate positive-signals in the mind and input that new data into the process-algorithm at step 18 by visualizing two or more principal thoughts about any objects-things, locations-spots, actions-items, or combinations that exist within or without, for example the ‘device on the nose or inhale-exhale of the breath and the device on the diaphragm or the movement of diaphragm’.

The method to induce sleep of the present invention will produce a useful, tangible, and concrete result for any person of any variety or heterogeneity of race with different sleeping habits, who has learned the skill of the art or the method. The positive-signals generated in the process to repair and transform brain-neurons, and produce a desired output is not a natural phenomenon as it can only be achieved using the method in the instant invention. The invention helps create bliss in mind and benefits any human-being who is unable to be free of intrusive thoughts streaming into the mind as his/her natural phenomena due to brain disorder.

Given the calming positive-signals as the new data input into the process-algorithm in the brain, the excited-signals from streaming intrusive thoughts are replaced, and the ‘repair and transformation’ of the brain-neurons into a different-physical state induces sleep as the new output. The beneficial result can be confirmed by the change in the recorded output of brain-activity when awake and while asleep.

The person using the method can sense the benefit or physical result after awakening from restful sleep. The same can be said of the user of sleeping-pills, except, the person who used drugs or pills gets up with a tired-drowsy feeling or headache.

The best mode to learn the method is for a person (of any variety or heterogeneity of race with vast difference in sleeping habits) to be willing to learn the art and perform the following steps: The person willing to learn is required to lay in bed (or sofa) with closed-eyes, and practice to visualize upon one principal thought at step 16, such as on the nose-band or inhalation and exhalation of breath (or one thought on other), until a calm mind is attained with decrease of intrusive thoughts in the mind. That practice should continue on the nose-band or inhalation and exhalation of breath or with different principal thoughts, one at a time, until one-thought which works consistently is identified.

The person willing to learn is required to continue step 18, and practice to visualize upon two or more principal thoughts calmly-equally at the same time, for example the device on the nose or inhalation-exhalation of the breath, and the device on the diaphragm or movement of diaphragm (or one set of thoughts on others), until a progressive tuning-in of signal-interference creates havoc in the brain's motor-responses, and splits the brain to make it fall asleep. That practice should continue with the device on the nose, inhalation-exhalation of the breath, the device on the diaphragm, movement of diaphragm, or using other principal thoughts, two or more at a time, until one-set of thoughts which works consistently is identified.

Any person who wants to be an artisan skilled in the art, or who wants to learn the use of the method for benefit, should get proper training to acquire the skill, because the method is not a natural process like the natural phenomenon of intrusive thoughts streaming into an insomniac brain due to anxiety, anger, depression or mental disorder. The method has no environmental and/or learning conditions provided the person is willing to learn.

A person willing to learn must identify the nature of intrusive thoughts in order to select appropriate principal-thoughts for practice in generating positive-signals in the mind, when the nose-band or the diaphragm-band do not yield a quick benefit. Such a finding can help select a set of objects-things, locations-spots, actions-items or combinations to use as principal thoughts in the practice of the method.

In this method to induce sleep, a person is required to learn to lay in bed (or sofa) and close eyes at the start of the step of concentrating on one-thought and keep them closed through the step of falling asleep to cut-off incoming light into the eyes and get dark-night's sleep. The person is required to learn to generate positive-signals in the mind and input that new data into the process-algorithm in the brain by visualizing one-principal thought by concentrating on the nose-band or inhalation-exhalation of the breath, or any object-thing, location-spot or action-item that exists within or without.

The person is then required to learn to generate positive-signals in the mind and input that new data into the process-algorithm in the brain by visualizing two or more principal thoughts, such as the nose-band or inhalation-exhalation of the breath, and the diaphragm-band or the movement of diaphragm, or any objects-things, locations-spots, actions-items, or combinations that exist within or without.

Any person who learns the method to induce sleep using the present invention can produce a useful, tangible, and concrete result whenever that person utilizes the process to decrease intrusive thoughts in the mind, and tune-in the signal-interference created havoc in the brain's motor-responses to split brain. The split brain transforms brain-neurons into a different-physical state to flip off the ‘light switch’ in the brain-stem. The brain having been repaired and transformed produces melatonin to invite sleep and serotonin to activate the master-clock in the brain, and begin a sleep-cycle. The result achieved in the instant invention is repeatable for practical application, and the final result produced is useful, tangible and concrete.

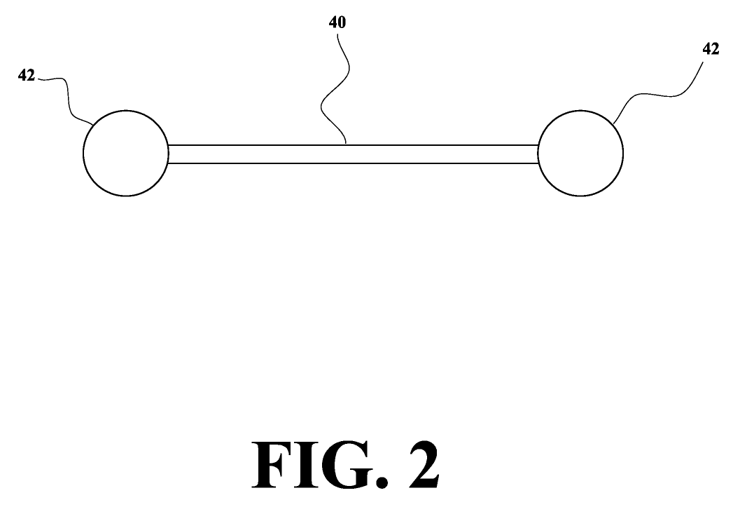

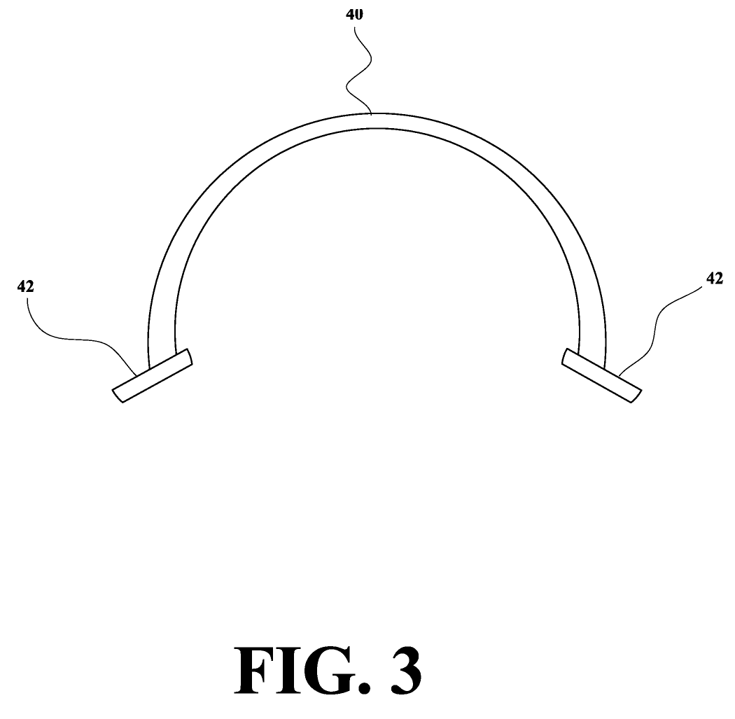

The device used in this method comes in two parts. The device to be worn on the nose is shown in FIGS. 2 and 3. It is made of a soft, flexible and elastic material. It is a band 40, of about ¼ inch-diameter (or wide) and 1½ inch-long (varies), configured to fit on to various nose-sizes, with self-adhesive plate-ends 42 (or self-vacuum cup-ends) of about ⅜ inch-diameter to stay stuck on to skin and keep it comfortably on the nose while in use. The nose-band is made from perforated (self-breathing) natural-rubber or equal, or any other suitable materials. The self-breathing device, with air passing out or in through perforations, elongates or contracts sufficiently to facilitate the user breathing air freely through the nose and deeply into the lungs. The nose-band is re-usable to help the user concentrate the mind on the device or inhalation-exhalation of the breath.

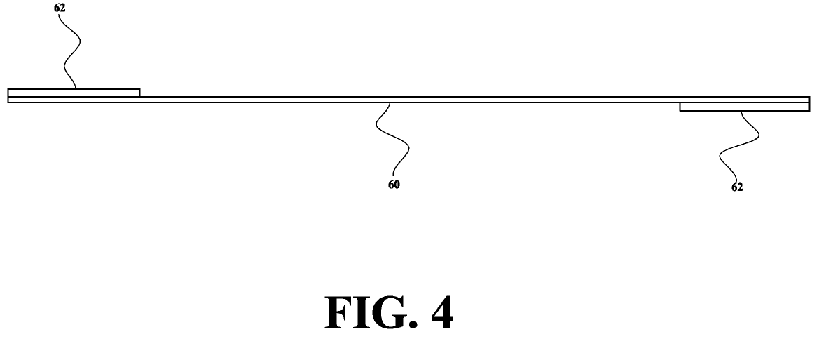

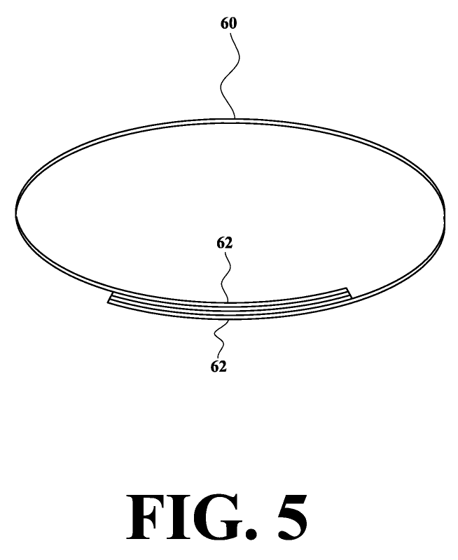

The other device used in this method, shown in FIGS. 4 and 5, is to be worn around the diaphragm. In FIG. 4 the diaphragm-band is shown in an open position, with the ends not joined. In FIG. 5 the diaphragm-band is shown in a closed position, with the ends joined, when it is worn around the diaphragm. It is made of a soft, flexible and elastic material. It is a band 60, of about ½ inch-diameter (or wide) and 24 to 48 inch-long (varies), configured to fit around various diaphragm-sizes, with ½ inch-wide and 3 to 6 inch-long self-adhesive ends 62, to stay stuck with one end on top of the other and keep it comfortably on the diaphragm while in use. The diaphragm-band is made from a perforated (self-breathing) natural rubber or equal, or any other suitable material. The self-breathing device, with air passing out or in through perforations, elongates or contracts sufficiently to facilitate the user breathing air freely through the nose and deeply into the lungs. The diaphragm-band is re-usable to help the user concentrate the mind on the device or movement of the diaphragm while inhaling and exhaling.

It is to be understood that the present invention is not limited to the sole embodiment described above, but encompasses any and all embodiments within the scope of the following claims.

I claim:

1. A method of inducing sleep, comprising the steps of:
(a) placing on the nose of a user a nose-band with adhesive end-cups that stick to the skin;
(b) placing a band over the diaphragm of the user, the diaphragm-band having two ends that overlap and can be joined together by self-adhesive;
(c) initiating tuning of the user's brain by concentrating the mind upon one principal thought, generating a set of positive-signals in the mind, inputting the positive-signals into neurons in the brain, tuning the new-signals replacing the old-signals in the brain, and decreasing the frequency of excited-signals in the cortex to calm the mind and cause the brain to produce melatonin; and
(d) continuing the tuning of the brain by concentrating the mind calmly and equally upon two or more principal thoughts at the same time, generating a set of positive-signals in the mind, inputting it into neurons in the brain, tuning the set of positive-signals to cause an interference created havoc in the brain's motor-responses, which splits the two hemispheres of the brain to stop them from communicating with each other, and makes the light switch in the brain-stem to flip off and cut off excited-signals coming into the brain, helping the brain produce serotonin to activate the master-clock and put the brain into a sleep-cycle.

2. The method of inducing sleep according to claim 1, wherein the user is required to lay down and close the user's eyes at the start of step (c) and keep them closed through step (d) to cut off incoming light into the eyes and get to sleep.

3. The method of inducing sleep according to claim 1, wherein the user generates positive-signals in the mind and inputs that new data into neurons in the brain at step (c) by visualizing one principal thought.

4. The method of inducing sleep according to claim 3, wherein the one principal thought is of an object.

5. The method of inducing sleep according to claim 3, wherein the one principal thought is of an action.

6. The method of inducing sleep according to claim 3, wherein the one principal thought is of a location.

7. The method of inducing sleep according to claim 1, wherein the user generates positive-signals in the mind and inputs that new data into neurons in the brain at step (d) by visualizing two or more principal thoughts.

8. The method of inducing sleep according to claim 7, wherein at least one of the principal thoughts is of an object.

9. The method of inducing sleep according to claim 7, wherein at least one of the principal thoughts is of an action.

10. The method of inducing sleep according to claim 7, wherein at least one of the principal thoughts is of a location.

11. The method of inducing sleep according to claim 1, wherein a user is being trained to practice and learn the method by a teacher.

12. The method of inducing sleep according to claim 11, wherein the user concentrates on a thought selected from the group comprising a thought on the nose-band and a thought on inhaling-exhaling of the breath.

13. The method of inducing sleep according to claim 11, wherein the user concentrates on a thought selected from a group comprising a thought on the diaphragm-band and a thought on the movement of the diaphragm.

14. The method of inducing sleep according to claim 11, wherein the user identifies the nature of intrusive thoughts causing an insomniac condition, in order to select appropriate principal-thoughts.

15. The method of inducing sleep according to claim 11, wherein the user concentrates on different principal thoughts, one or more at a time, until one or more thoughts which work consistently are identified.

16. The method of inducing sleep according to claim 1, wherein the nose-band is made of a soft, flexible and elastic material, and is suitably dimensioned and configured to fit onto the nose of the user.

17. The method of inducing sleep according to claim 16, wherein the soft, flexible and elastic material is perforated rubber.

18. The method of inducing sleep according to claim 1, wherein the diaphragm-band is made of a soft, flexible and elastic material, and is suitably dimensioned and configured to fit over the diaphragm of the user.

19. The method of inducing sleep according to claim 18, wherein the soft, flexible and elastic material is perforated rubber.

20. The method of inducing sleep according to claim 1, wherein the nose-band and the diaphragm-band are sufficiently elastic that the ends stay stuck and keep in position while in use for breathing air freely through the nose and deeply into the lungs.
